(19) United States
(12) Patent Application Publication
(10) Pub. No.: US 2012/0330275 A1
(43) Pub. Date: Dec. 27, 2012

(54) Syringe Evacuator

(75) Inventors: Malcolm Rude, College Station, TX (US); Brian Adler Mirth, College Station, TX (US)

(73) Assignee: Medinvention LLC, College Station, TX (US)

(21) Appl. No.: 13/167,583

(22) Filed: Jun. 23, 2011

(51) Int. Cl. A61M 5/315

(52) U.S. Cl. 604/506; 604/211

(57) ABSTRACT

A syringe evacuator configured to discharge a syringe at a controllable rate. In one embodiment, the syringe evacuator includes a threaded shaft and a syringe depressor platform threaded onto the threaded shaft. The syringe evacuator also includes a spiral spring coupled to the threaded shaft configured to rotate the threaded shaft.

### TECHNICAL FIELD

The present invention is directed, in general, to a syringe evacuator that produces a consistent and controllable rate of evacuation of the contents of a syringe.

### BACKGROUND

In the medical field, there is a need to evacuate syringes at a controlled rate. Various techniques have been proposed to perform this function including motorized pumps, pneumatic drives and coil springs. Motors and pneumatic drives have the potential for delivering a consistent force, but have the disadvantage of increased complexity, expense, and the need for an electrical or compressed air power source. Coil springs have been used to depress syringe plungers, but exert significantly different forces when fully compressed and minimally compressed. There are numerous extant spring-driven syringe devices, but such devices lack the ability to give a steady flow rate. The current technology has predictably varying flow depending on the compression of a spring.

A reusable and adjustable syringe evacuator that enables its contents to be injected at a consistent and controllable rate is needed in certain applications such as surgical techniques for cosmetic and reconstruction purposes. For example, fat injection has a wide range of clinical cosmetic and reconstruction benefits wherein it is necessary that a syringe filled with a fat-containing solution or more generally a medicine, be emptied at a consistent rate. The device that performs such evacuation should be a self-contained and a reusable design that does not require the use of an electrical motor or compressed air to deliver a consistent force to the contents of the syringe. The device should be easy to use and amenable to ergonomic design.

Accordingly, what is needed in the art is a syringe evacuator that enables its contents to be evacuated at a consistent and controllable rate that overcomes deficiencies in the prior art.

### SUMMARY OF THE INVENTION

These and other problems are generally solved or circumvented, and technical advantages are generally achieved, by advantageous embodiments of the present invention, including a syringe evacuator configured to discharge a syringe at a controllable rate. In one embodiment, the syringe evacuator includes a threaded shaft and a syringe depressor platform threaded onto the threaded shaft. The syringe evacuator also includes a spiral spring coupled to the threaded shaft configured to rotate the threaded shaft.

The foregoing has outlined rather broadly the features and technical advantages of the present invention in order that the detailed description of the invention that follows may be better understood. Additional features and advantages of the invention will be described hereinafter, which form the subject of the claims of the invention. It should be appreciated by those skilled in the art that the conception and specific embodiment disclosed may be readily utilized as a basis for modifying or designing other structures or processes for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present invention, reference is now made to the following descriptions taken in conjunction with the accompanying drawings, in which:

FIGS. 1, 2A and 2B illustrate an isometric view, a side view and a top view, respectively, of an embodiment of a syringe evacuator fitted with a syringe constructed according to the principles of the present invention;

FIGS. 3 and 4 illustrate an isometric view and a side view, respectively, of an embodiment of a syringe evacuator constructed according to the principles of the present invention;

FIGS. 5, 6 and 7 illustrate a bottom view, a cross sectional view and an exploded view, respectively, of an embodiment of a syringe evacuator or portions thereof constructed according to the principles of the present invention;

FIGS. 8A and 8B illustrate a side view and a partially unassembled view, respectively, of an embodiment of a syringe evacuator constructed according to the principles of the present invention;

FIGS. 9 and 10 illustrate end views of an embodiment of a portion of a syringe evacuator constructed according to the principles of the present invention; and

FIG. 11 illustrates a flow chart of an embodiment of a method of forming a syringe evacuator according to the principles of the present invention; and

FIG. 12 illustrates a flow chart of an embodiment of a method of operating a syringe evacuator according to the principles of the present invention.

Corresponding numerals and symbols in the different figures generally refer to corresponding parts unless otherwise indicated, and may not be redescribed in the interest of brevity after the first instance. The FIGUREs are drawn to illustrate the relevant aspects of exemplary embodiments.

### DETAILED DESCRIPTION OF ILLUSTRATIVE EMBODIMENTS

The making and using of the present exemplary embodiments are discussed in detail below. It should be appreciated, however, that the present invention provides many applicable inventive concepts that can be embodied in a wide variety of specific contexts. The specific embodiments discussed are merely illustrative of specific ways to make and use the invention, and do not limit the scope of the invention.

The present invention will be described with respect to exemplary embodiments in a specific context, namely, a syringe evacuator that produces a substantially consistent and controllable rate of evacuation (or discharge) of the contents of a syringe and methods of forming and operating the same. While the principles of the present invention will be described in the environment of a medical procedure, any application that may benefit from a syringe evacuator is well within the broad scope of the present invention.

There are numerous applications in medicine such as dispensing medicines or fat injection during fat grafting that would benefit from a controllable rate evacuation syringe. Certain medications are very sensitive to the injected rate of flow. Examples of such medications are the anesthetic Propofol® and the antibiotic Vancomycin®, which if given too rapidly, produce serious complications. (See, e.g., Sivagnanam, S. and Deleu, D. in an article entitled “Red Man Syndrome,” published in the journal Critical Care (London, England) 7, pp. 119-120, 2003, which is incorporated herein by reference.) Electric pumps have been used previously to deliver medicines at a consistent rate such as for the administration of some intravenous medications.

Fat injection has a wide range of clinical applications for cosmetic and reconstructive purposes as described in the journal Plastic & Reconstructive Surgery, volume 124—issue 1, pp 272-280, July 2009, which is incorporated herein by reference. The technique of fat injection introduces small aliquots of fat that are revascularized by the recipient bed. The injection is typically done with manual pressure to depress a syringe plunger. Particularly for large volumes of fat injection, there is an unmet need to have a simple way to evacuate the fat from the syringe at a consistent rate. There are numerous applications that can benefit from consistent delivery of the contents of a syringe including, but not limited to, fat for grafting and medicine applications.

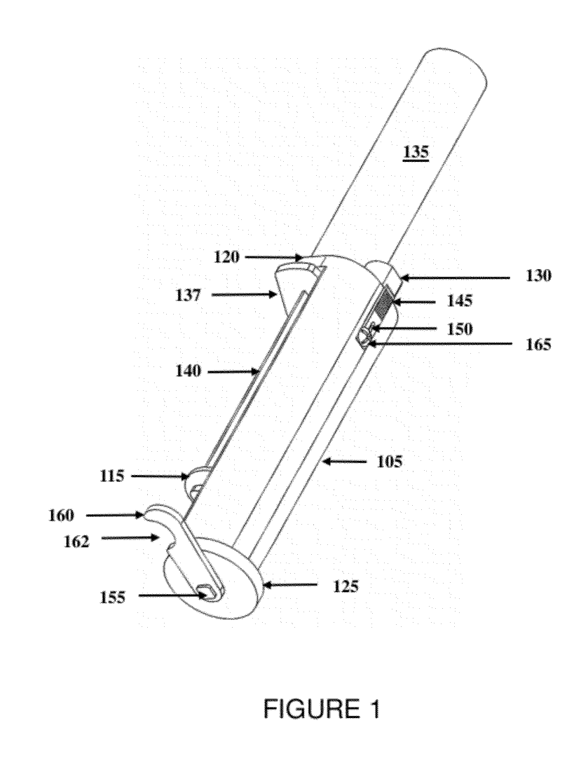

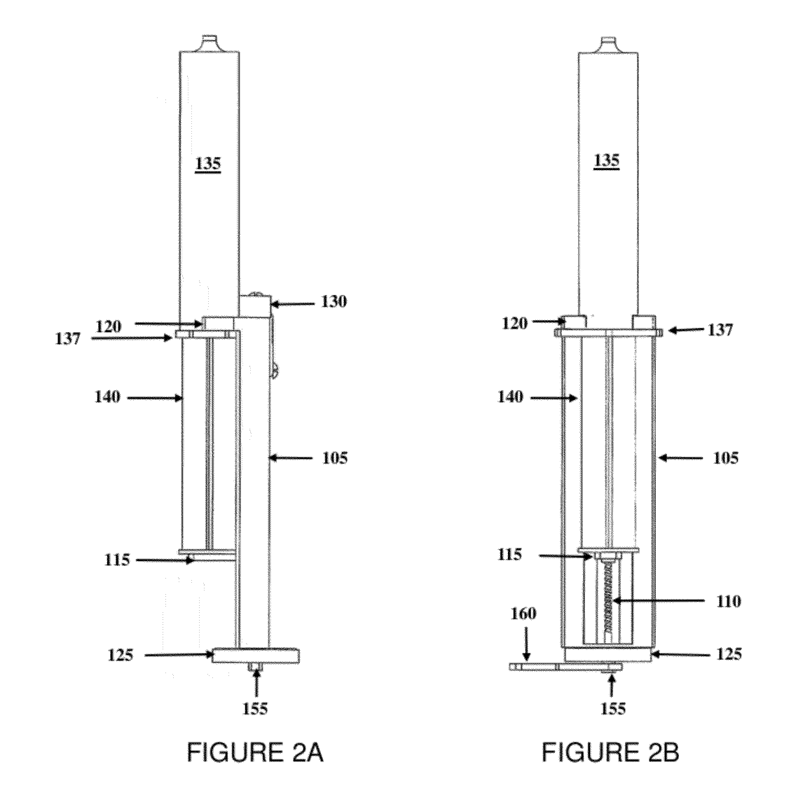

Referring initially to FIGS. 1, 2A and 2B, illustrated are an isometric view, a side view and a top view, respectively, of an embodiment of a syringe evacuator 105 fitted with a syringe including a syringe body 135 and a syringe plunger 140 constructed according to the principles of the present invention. The syringe evacuator 105 applies a substantially consistent pressure to the syringe plunger 140 to produce controllable evacuation (or discharge) of the contents of the syringe at a substantially consistent rate that can be varied and manually adjusted. A standard, fully loaded syringe is inserted in the syringe evacuator 105 with the syringe plunger 140 between an open clamp 120 and a syringe depressor platform 115 of the syringe evacuator 105. The syringe is secured to the open clamp 120 at the syringe hub 137.

With the syringe depressor platform 115 in a substantially fully retracted position, the syringe can be fully loaded, without limitation, with medicine or a fat-containing solution. The syringe plunger 140 contacts the syringe depressor platform 115. The syringe depressor platform 115 can be placed in a less retracted position by depressing a control lever 145, allowing a spiral spring enclosed in a spiral spring housing 125 to rotate a threaded shaft 110. The control lever 145 provides the ability to start and stop flow from the syringe and is held in place by a retaining device 165 (e.g., a screw). There is a slit 150 in the body of the control lever 145 that acts as a spring to provide a force to press a brake pad of the control lever 145 against the brake drum 130.

A wrench 160 of the syringe evacuator 105 may be employed to rotate a mechanical structure (e.g., a nut 155) that rotates the threaded shaft 110 to rewind the spiral spring enclosed in the spiral spring housing 125 thereby allowing the syringe evacuator 105 to be reusable. There is a one-sided opening on the wrench 160 for the nut 155 to fit. A finger groove 162 on the wrench 160 is designed to inform a user the direction to rewind the spiral spring.

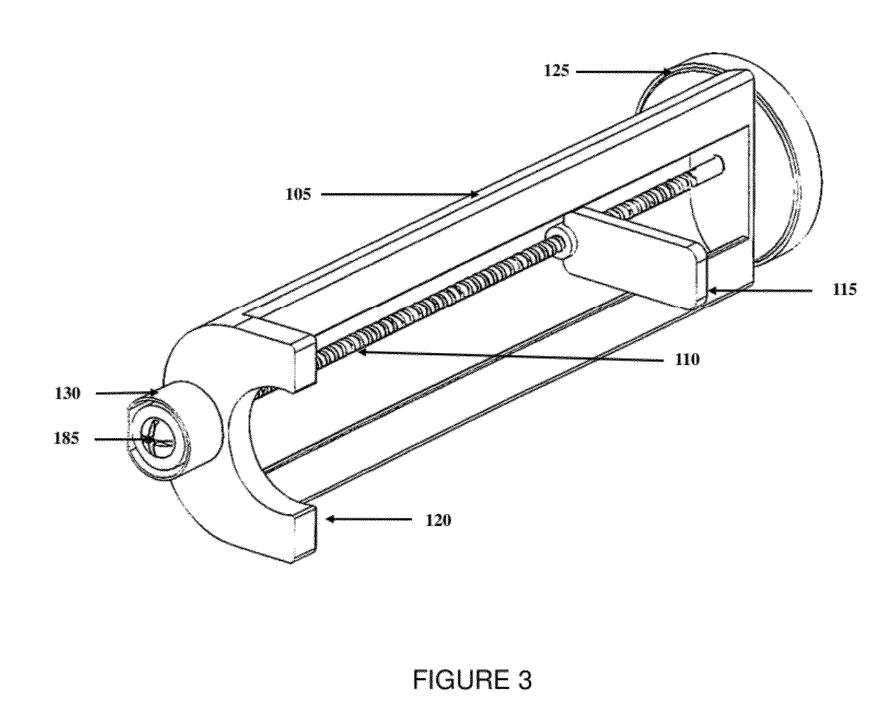

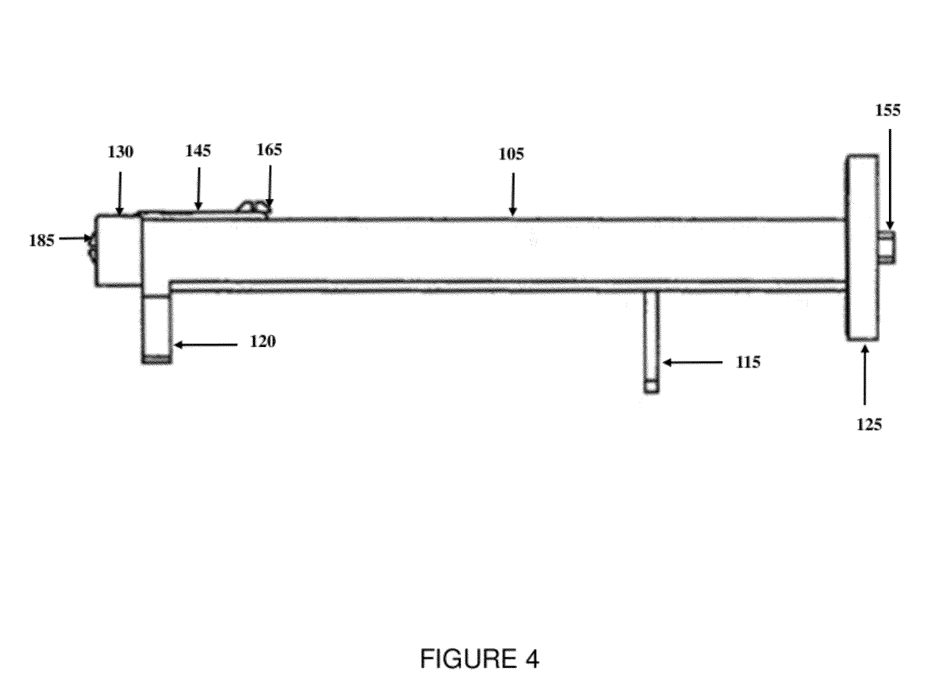

Turning now to FIGS. 3 and 4, illustrated are an isometric view and a side view, respectively, of an embodiment of a syringe evacuator constructed according to the principles of the present invention. For purposes of the discussion herein, similar features to the syringe evacuator of the preceding FIGUREs will have the same reference designations. Thus, as mentioned above, the syringe evacuator 105 includes a brake drum 130 (which is also shown in FIGS. 1 and 2A) and a retaining device 185 (e.g., a screw). As described herein, the flow rate of the contents of a syringe may be varied by manually operating the control lever 145 that applies an adjustable friction force to the brake drum 130.

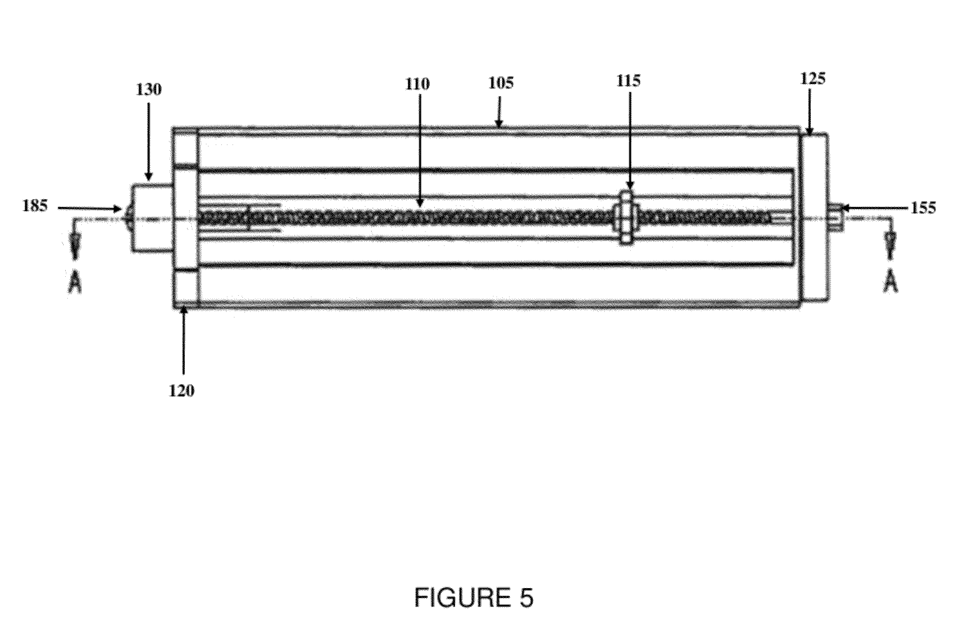

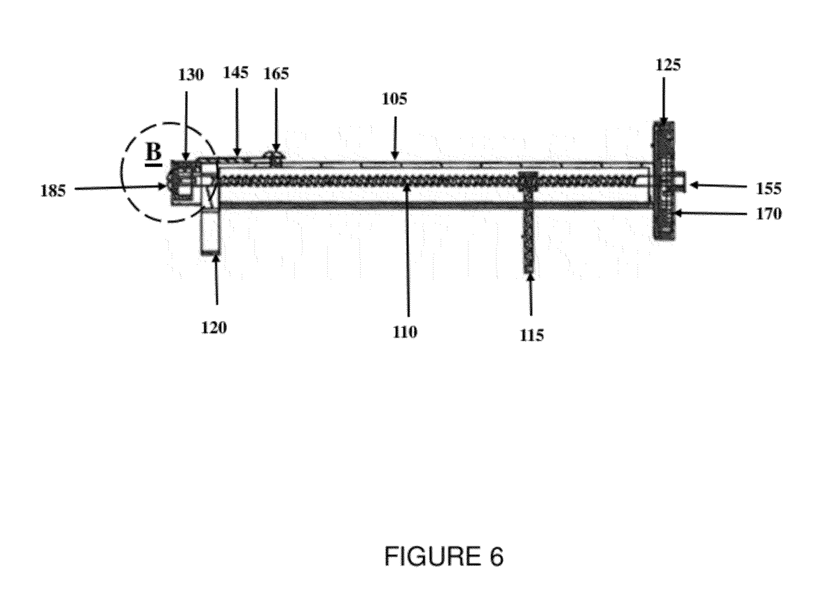

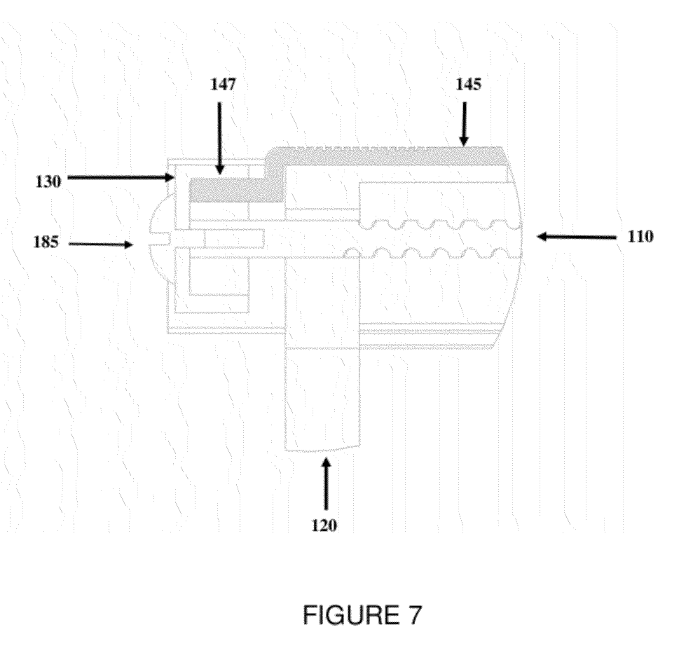

Turning now to FIGS. 5, 6 and 7, illustrated are a bottom view, a cross sectional view and an exploded view, respectively, of an embodiment of a syringe evacuator or portions thereof constructed according to the principles of the present invention. More specifically, FIG. 6 is a cross sectional view along line A-A of FIG. 5 and FIG. 7 is an exploded view of the brake drum 130 designated detail B in FIG. 6. Again, for purposes of the discussion herein, similar features to the syringe evacuator of the preceding FIGUREs will have the same reference designations.

The syringe evacuator 105 has capacity for varying the flow rate by adjusting tension of a spiral spring 170 by rotating the spiral spring housing 125, or to lock it at a maximum flow. The flow rate may also be varied by manually operating the control lever 145 that applies an adjustable friction force via a brake pad 147 to the brake drum 130. The syringe evacuator 105 may be reused by rewinding the spiral spring 170 via the nut 155, thereby proximally repositioning the syringe depressor platform 115.

The syringe is evacuated by action of the syringe depressor platform 115 against the syringe plunger 140. The spiral spring 170 provides a substantially consistent force to rotate the threaded shaft 110 to advance the syringe depressor platform 115. A spirally shaped spring was chosen for its capacity to deliver a substantially consistent, relatively unvarying force through the central portion of its compression. As the spiral spring 170 rotates the threaded shaft 110, the syringe depressor platform 115 is advanced distally to evacuate the syringe. This mechanism allows the syringe depressor platform 115 to be depressed at a substantially consistent and controllable rate. The spiral spring 170 provides the syringe evacuator drive mechanism. Conventional syringe evacuation devices apply pressure to a syringe plunger with a coil spring. As introduced herein, a spiral spring is employed to deliver a substantially consistent force to a syringe discharging mechanism as it releases a portion of its tension with relatively little torque variation.

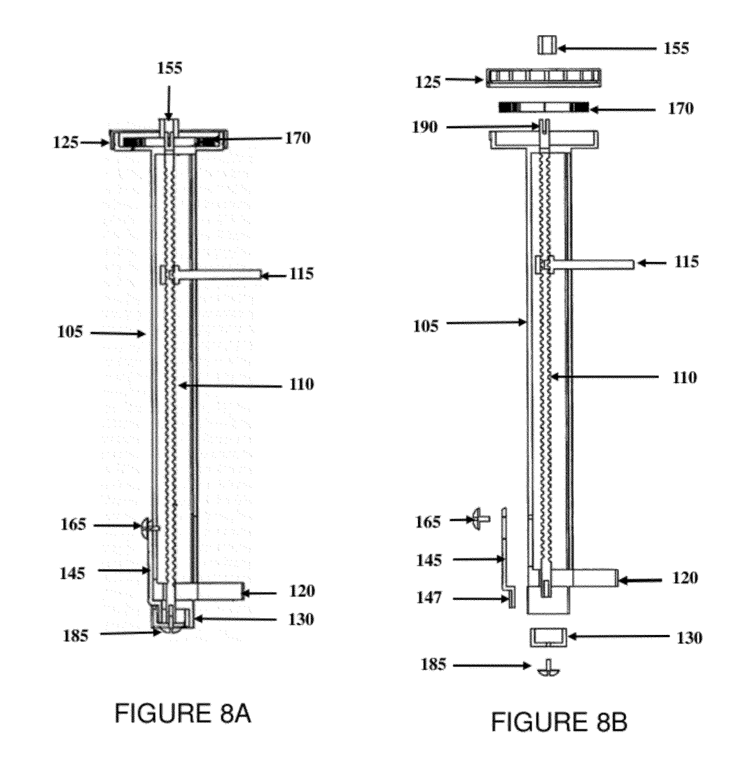

Turning now to FIGS. 8A and 8B, illustrated are a side view and a partially unassembled view, respectively, of an embodiment of a syringe evacuator constructed according to the principles of the present invention. Again, for purposes of the discussion herein, similar features to the syringe evacuator of the preceding FIGUREs will have the same reference designations. As mentioned above, the spiral spring 170 is housed in a spring chamber covered by rotatable spiral spring housing 125. As more clearly shown in FIG. 8B, the innermost end of the spiral spring 170 is fitted into a slot 190 at the proximal end of the threaded shaft 110.

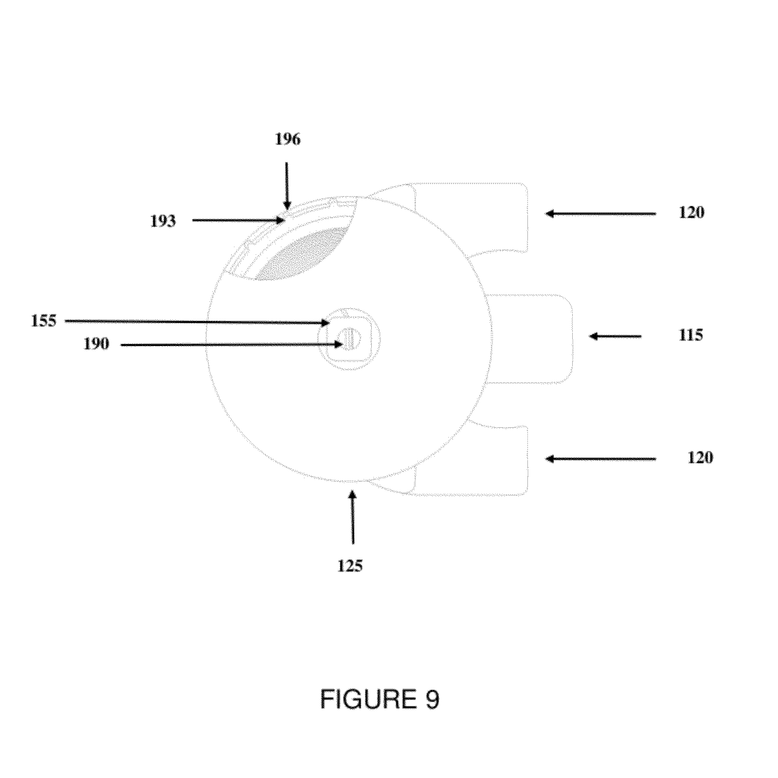

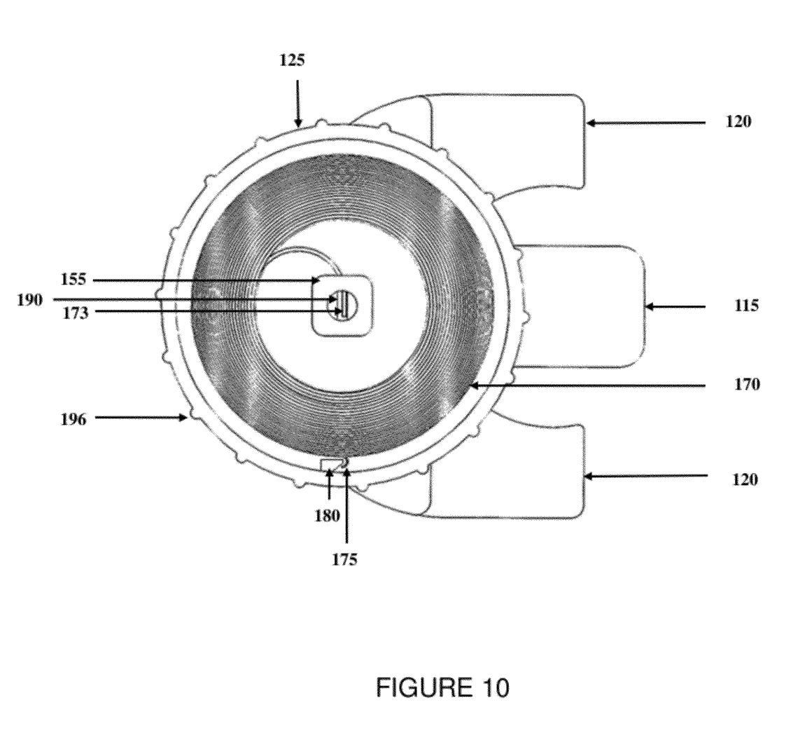

Turning now to FIGS. 9 and 10, illustrated are end views of an embodiment of a portion of a syringe evacuator constructed according to the principles of the present invention. Again, for purposes of the discussion herein, similar features to the syringe evacuator of the preceding FIGUREs will have the same reference designations. In particular, FIG. 9 illustrates an end view with a portion of a cover of the spiral spring housing 125 removed and FIG. 10 illustrates an end view with the cover of the spiral spring housing 125 entirely removed. As mentioned above, the spiral spring 170 is housed in a spring chamber covered by rotatable spiral spring housing 125. The innermost end 173 of the spiral spring 170 is fitted into a slot 190 at the proximal end of the threaded shaft 110. The outermost end 175 of the spiral spring 170 is bent into a formed end so that it can be restrained by a ridge or tab 180 on the inside of the spiral spring housing 125, as illustrated in FIG. 10.

The level of torque generated by the spiral spring 170 can be varied by tightening or loosening the spiral spring 170 by rotating the spiral spring housing 125 to adjust the force exerted on the syringe plunger 140 (see FIGS. 1, 2A and 2B) to produce varying (but substantially consistent) rates of discharge of the contents of the syringe. The ridge or tab 180 anchors the outermost end 175 of the spiral spring 170 to the spiral spring housing 125. Tension of the spiral spring 170 is increased by rotating the spiral spring housing 125 (e.g., clockwise). Tension of the spiral spring 170 is decreased by rotating the spiral spring housing 125 in the opposite direction (e.g., counterclockwise). Ribs 193 on the spiral spring chamber articulate with grooves 196 on the inner surface of the spiral spring housing 125. The ribs 193 allow the spring tension adjustment to stay in place during use and after being turned to loosen or tighten spiral spring 170. This articulation provides sufficient friction to overcome the torque of the spiral spring 170. The tension of the spiral spring 170 can be changed to control the flow rate of the contents of the syringe depending on the desired speed of evacuation in view of the viscosity of the contents of the syringe that is being evacuated.

With continuing reference to the preceding FIGUREs, the threaded shaft 110 can be held in a static position by friction of brake drum 130 against the brake pad 147 of control lever 145. Depression of control lever 145 releases the brake pad 147 of the control lever 145 and the rubber brake drum 130. This allows for manual on and off control of flow of the contents of the syringe as well as manual control to vary the rate of flow thereof. The control lever 145 may also be withdrawn proximally to allow the spiral spring 170 to drive the threaded shaft 110 at a maximal rate given the tension of the spiral spring 170.

The restraining device 165 inserts into the slit 150 in the body of the control lever 145. This allows the control lever 145 to be held in a withdrawn position with the brake drum 130 fully released. When slightly depressed, friction of the control lever 145 with brake drum 130 is decreased allowing variable speed control of threaded shaft 110. When at rest, control lever 145 is in a position to stop movement of the threaded shaft 110. When the control lever 145 is fully depressed or pushed back into a locked on position, the threaded shaft 110 has no resistance from the brake drum 130.

The syringe depressor platform 115 can be returned to its proximal position and the spiral spring 170 can be rewound by applying the rewinding wrench 160 to the nut 155. A triangular brake pad 147 of the control lever 145 may provide sufficient friction to overcome the force of the spiral spring 170, but the friction of the brake pad 147 of the control lever 145 can be overcome by the force of rewinding the spiral spring 170 at the nut 155. The spiral spring housing 125 is formed with the triangular ridge or tab 180 to restrain the spiral spring 170. The innermost end 173 of the spiral spring 170 fits into the slot 190 in an end of threaded shaft 110 to engage the spiral spring 170 in the proximal end of the threaded shaft 110. The outermost end 175 of the spiral spring 170 is held by the ridge or tab 180 on the inside of the spiral spring housing 125. The spiral spring housing 125 has an opening on one side to insert the nut 155. As mentioned above, a finger groove 162 on the wrench 160 is designed to inform the user the direction to rewind the spiral spring.

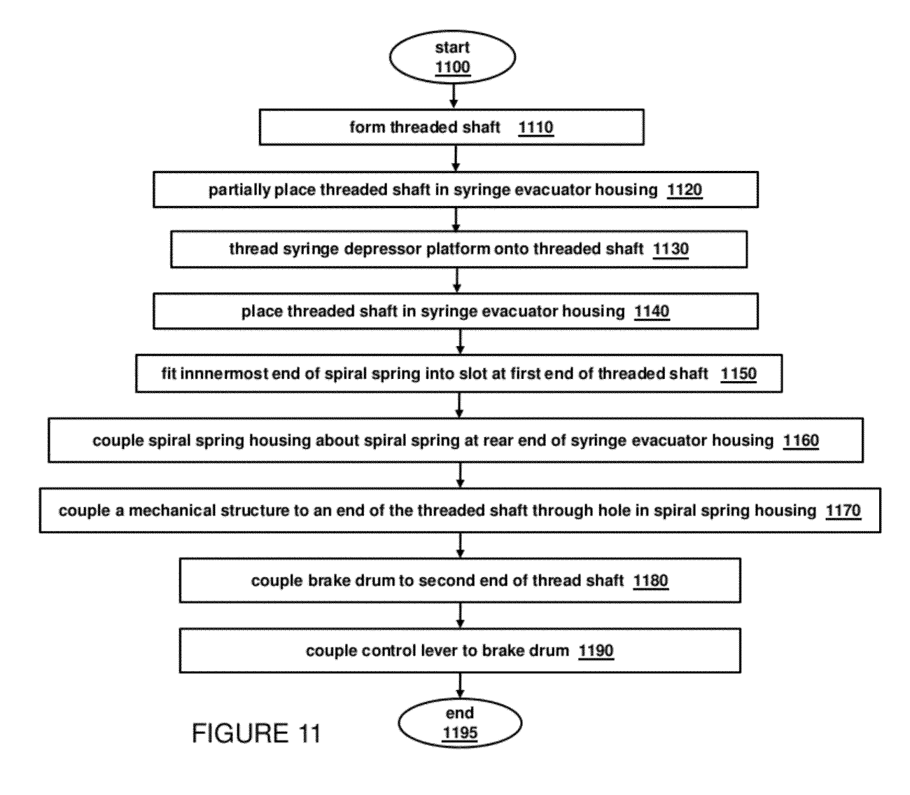

Turning now to FIG. 11, illustrated is a flow chart of an embodiment of a method of forming a syringe evacuator according to the principles of the present invention. The method begins in a step or module 1100 to construct a syringe evacuator. In a step or module 1110, a threaded shaft is formed with a slot at a first end thereof. In a step or module 1120, the threaded shaft is partially placed in a syringe evacuator housing with a second end of the threaded shaft being threaded through an opening in a rear end of the syringe evacuator housing. In a step or module 1130, a syringe depressor platform is threaded onto the second end of the threaded shaft between a fore end and the rear end of the syringe evacuator housing. In a step or module 1140, the threaded shaft is further placed in the syringe evacuator housing by continuing to thread the threaded shaft through the opening in the rear end of the syringe evacuator housing until the second end of the threaded shaft goes through an opening of an open clamp at the fore end of the syringe evacuator housing. The open clamp is configured to restrain a forward motion of a syringe positioned in the syringe evacuator. Thus, the syringe depressor platform is threaded onto the threaded shaft with a first end of the threaded shaft coupled to the rear end of the syringe evacuator housing and a second end of the threaded shaft coupled to the fore end of the syringe evacuator housing.

In a step or module 1150, an innermost end of a spiral spring is fitted into the slot at the first end of the threaded shaft to allow the spiral spring to rotate the threaded shaft. In a step or module 1160, a rotatable spiral spring housing is coupled to the rear end of the syringe evacuator housing about and to provide tension to the spiral spring. In accordance therewith, an outermost end of the spiral spring is restrained by a ridge or tab on the inside of the spiral spring housing. The spiral spring is configured to discharge a syringe held in the syringe evacuator at a substantially consistent rate. In a step or module 1170, a mechanical structure such as a nut is coupled to an end of the threaded shaft through an opening in the spiral spring housing to enable the threaded shaft to be manually rotated.

In a step or module 1180, a brake drum is coupled to the second end of the threaded shaft. In a step or module 1190, a control lever is coupled to the syringe evacuator housing and the brake drum to apply an adjustable friction force thereto. The control lever is configured to adjust a discharge rate of a syringe retained in the syringe evacuator. The brake drum may be secured about a brake pad of the control lever once the control lever is attached to the syringe evacuator housing. The method ends at step or module 1195.

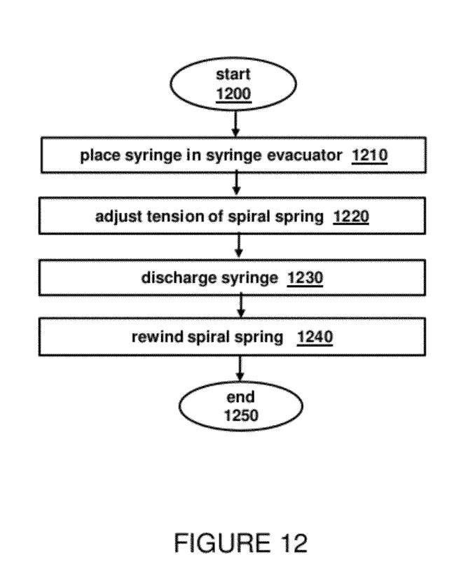

Turning now to FIG. 12, illustrated is a flow chart of an embodiment of a method of operating a syringe evacuator according to the principles of the present invention. The method begins in a step or module 1200 to operate a syringe evacuator. In a step or module 1210, a syringe is placed in a syringe evacuator by securing a syringe plunger between an open clamp and a syringe depressor platform of the syringe evacuator. In a step or module 1220, a tension of a spiral spring coupled to a threaded shaft is adjusted by rotating a spiral spring housing of the syringe evacuator. In a step or module 1230, the syringe depressor platform is depressed against the syringe plunger to discharge the syringe, preferably at a substantially consistent rate. For whatever reason, if the discharge rate needs to be changed, the tension of the spiral spring coupled to the threaded shaft may be adjusted in accordance with a control lever cooperating with a brake drum. After the contents of the syringe are discharged, in a step or module 1240, the threaded shaft may be manually rotated with a mechanical structure coupled to the threaded shaft to rewind the spiral spring, thereby allowing the syringe evacuator to be reusable. The method ends at step or module 1250.

Thus, a syringe evacuator has been introduced herein configured to discharge a syringe at a controllable rate. In one embodiment, the syringe evacuator includes a threaded shaft and a syringe depressor platform threaded onto the threaded shaft. The syringe evacuator also includes a spiral spring coupled to the threaded shaft configured to rotate the threaded shaft. The syringe evacuator may also include a control lever configured to apply an adjustable friction force to a brake drum to adjust a discharge rate of a syringe retained in said syringe evacuator. The syringe evacuator may also include a mechanical structure (e.g., a nut) coupled to an end of the threaded shaft configured to enable the threaded shaft to be manually rotated, thereby allowing the syringe evacuator to be reusable. The syringe evacuator may also include an open clamp configured to restrain a forward motion of a syringe to be positioned in the syringe evacuator. The syringe evacuator may also include a spiral spring housing rotatably coupled to the syringe evacuator configured to provide tension to the spiral spring via a tab configured to engage a formed end of the spiral spring. The spiral spring is configured to discharge a syringe held in the syringe evacuator at a substantially consistent rate.

Those skilled in the art should understand that the previously described embodiments of a syringe evacuator and related methods of constructing the same are submitted for illustrative purposes only. In addition, other embodiments capable of producing a controlled rate of evacuation of a syringe employable in other applications are well within the broad scope of the present invention. While the syringe evacuator has been described in the environment of injecting a medicine or fat-containing solution it may also be applied to other systems such as a system employed to execute a chemical procedure.

Also, although the present invention and its advantages have been described in detail, it should be understood that various changes, substitutions and alterations can be made herein without departing from the spirit and scope of the invention as defined by the appended claims. For example, many of the processes discussed above can be implemented in different methodologies and replaced by other processes, or a combination thereof.

Moreover, the scope of the present application is not intended to be limited to the particular embodiments of the process, machine, manufacture, composition of matter, means, methods, and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A syringe evacuator, comprising:
a threaded shaft;
a syringe depressor platform threaded onto said threaded shaft; and
a spiral spring coupled to said threaded shaft configured to rotate said threaded shaft.

2. The syringe evacuator as recited in claim 1 further comprising a brake drum coupled to said threaded shaft.

3. The syringe evacuator as recited in claim 2 further comprising a control lever configured to apply an adjustable friction force to said brake drum.

4. The syringe evacuator as recited in claim 3 wherein said control lever is configured to adjust a discharge rate of a syringe retained in said syringe evacuator.

5. The syringe evacuator as recited in claim 1 further comprising a mechanical structure coupled to an end of said threaded shaft configured to enable said threaded shaft to be manually rotated.

6. The syringe evacuator as recited in claim 5 wherein said mechanical structure comprises a nut.

7. The syringe evacuator as recited in claim 1 further comprising an open clamp configured to restrain a forward motion of a syringe to be positioned in said syringe evacuator.

8. The syringe evacuator as recited in claim 1 further comprising a spiral spring housing rotatably coupled to said syringe evacuator configured to provide tension to said spiral spring.

9. The syringe evacuator as recited in claim 8 wherein said spiral spring housing comprises a tab configured to engage a formed end of said spiral spring.

10. The syringe evacuator as recited in claim 1 wherein said spiral spring is configured to discharge a syringe held in said syringe evacuator at a substantially consistent rate.

11. A method, comprising:
placing a syringe in a syringe evacuator by securing a syringe plunger between an open clamp and a syringe depressor platform of said syringe evacuator;
adjusting a tension of a spiral spring coupled to a threaded shaft by rotating a spiral spring housing of said syringe evacuator; and
depressing said syringe depressor platform against said syringe plunger to discharge said syringe.

12. The method as recited in claim 11 further comprising adjusting a discharge rate of said syringe.

13. The method as recited in claim 11 further comprising manually rotating said threaded shaft to rewind said spiral spring.

14. The method as recited in claim 11 wherein said syringe is discharged at a substantially consistent rate.

15. A method, comprising:
providing a threaded shaft;
placing said threaded shaft in a syringe evacuator housing;
threading a syringe depressor platform onto said threaded shaft; and
coupling a spiral spring to an end of said threaded shaft.

16. The method as recited in claim 15 further comprising coupling a brake drum coupled to another end of said threaded shaft.

17. The method as recited in claim 16 further comprising coupling a control lever to said brake drum.

18. The method as recited in claim 15 further comprising coupling a mechanical structure to said end of said threaded shaft configured to enable said threaded shaft to be manually rotated.

19. The method as recited in claim 15 wherein said syringe evacuator housing comprises an open clamp configured to restrain a forward motion of a syringe to be positioned in said syringe evacuator.

20. The method as recited in claim 15 further comprising coupling a spiral spring housing to said end of said threaded shaft about said spiral spring.
